(19) United States
(12) Patent Application Publication
(10) Pub. No.: US 2021/0208135 A1
(43) Pub. Date: Jul. 8, 2021

(54) INKJET-PRINTED ELECTROCHEMICAL METABOLITE SENSORS

(71) Applicant: King Abdullah University of Science and Technology, Thuwal (SA)

(72) Inventors: Sahika Inal, Thuwal (SA); Eloise Bihar, Thuwal (SA)

(21) Appl. No.: 17/058,073

(22) PCT Filed: Apr. 29, 2019
(86) PCT No.: PCT/IB2019/053496

§ 371 Date: Nov. 23, 2020

Related U.S. Application Data
(60) Provisional application No. 62/674,374, filed on May 21, 2018.

(51) Int. Cl. G01N 33/543; G01N 27/327

(52) U.S. Cl. CPC G01N 33/5438; G01N 27/3272

(57) ABSTRACT

Described are inkjet-printed sensors for detecting metabolites in biological samples obtained non-invasively. The sensors may include a backing layer, and at least one set of three electrodes printed from a conducting polymer onto the backing layer. A set of electrodes includes a three-electrode geometry with a reference electrode, a working electrode with a polymeric coating, and a counter electrode. The sensor may be connected to an acquisition system and/or a display system, forming a sensor system. The biological sample may be saliva, sputum, tear, sweat, urine, exudate, blood, plasma, or vaginal discharge. The sensor typically detects metabolites capable of interacting with oxidase or oxido-reductase enzymes. Some of the exemplary metabolites detected by the sensor include glucose, cholesterol, nicotine, carbon monoxide, nitrite, nitrate, alcohol, and bacterial metabolites.

### CROSS-REFERENCE TO RELATED APPLICATION

This application claims the benefit of and priority to U.S. Ser. No. 62/674,374 filed May 21, 2018, which is incorporated by reference in its entirety.

### FIELD OF THE INVENTION

The invention is generally directed to portable metabolite sensors for detecting metabolites in biological samples obtained non-invasively.

### BACKGROUND OF THE INVENTION

Diabetes and cardiovascular diseases are still the major causes of mortality worldwide, with diabetes particularly affecting more than 425 million people every year (Estimated number diabetics worldwide 2017). Diabetes is tightly linked with uncontrolled blood sugar levels which lead to a host of complications and damages in vital organs. Current metabolite detection methods typically require instrumentation and/or multiple handling steps (i.e., enzyme-linked immunosorbent assay (ELISA)-based kits) which renders them not very convenient to use and definitely not near-patient testing devices

Direct access to low cost, minimally invasive diagnostic tests which eliminate the need of trained personnel is a major prerequisite to broadening access to and participation in preventative healthcare. Tests that allow self-monitoring and one-shot use are imperative, particularly in low-resource settings where, in most cases, neither sufficient infrastructure nor personnel exist (Sharma, et al., Biosensors, 5:577-601 (2015)). While screening blood for irregularities is typically performed as part of a routine checkup in developed countries, measurement of blood glucose is accessible only in about 50% of the primary care settings in low income countries (Wang, et al., J. Food Drug Anal., 23:191-200 (2015)). Additionally, the use of a whole-blood sample introduces further limitations, for example, limitations in patient compliance as a result of the pain and inconvenience associated with multiple needle pricks, and secondly the trained personnel required to perform the tests in some cases. The fabrication of traditional “hard” electronics typically involves tedious and expensive processing methods (such as lithography) due to the nature of the electrode materials used.

Therefore, there is a high demand and need for self-monitoring of critical biomarkers in the body using biofluids alternative to blood, such as saliva and sweat (Soni, et al., Biosens. Bioelectron., 67:763-768 (2015); Pappa, et al., Adv. Healthc. Mater., 5:2295-2302 (2016); Lee, et al., Adv. Healthc. Mater., 7:1701150-1701164 (2018); Gao, et al., Nature, 529:509-514 (2016); and Lee, et al., Sci. Adv., 3:e1601314 (2017)). With the evolution of microelectronics and the possibility of automation, electrochemical sensors have been developed for detecting glucose and for real-world applicability, e.g. wearables (Wang, Chem. Rev., 108:814-825 (2008); Pappa, et al., Trends Biotechnol., 36:45-49 (2017); and Kim, et al., Talanta, 177:163-170 (2018)). The majority of current electrochemical metabolite sensors rely on the function of enzymes (oxidoreductases) (Wang, Electroanalysis, 17:7-14 (2005) and Grieshaber, et al., Sensors, 8:1400-1458 (2008)).

For the fabrication of sensors wherein application demands are not strictly driven by performance but also by cost-efficiency, for instance those that are designed to be disposable and for single use, additive printing techniques (e.g., inkjet and screen printing) and materials compatible with large area deposition have been employed. For instance, use of organic electrochemical transistors for the detection of multi-metabolites using saliva as a media via the separate biofunctionalization of transistor with specific enzyme was reported by Pappa et al., Adv. Healthc. Mater. 5:2295-2302 (2016). Inkjet-printing allows for the controlled deposition of a variety of electronic materials in customized geometries, constitutes a low temperature process (Calvert, Chem. Mater., 13:3299-3305 (2001)) and can be performed in a single step. However, there is still a need for biosensor devices produced completely by methods such as inkject printing, thus providing devices which are superior in terms of cost-effectiveness and affordability, and which can detect metabolite in a minimally invasive manner There is also a need for devices whose components are all produced by cost-effective means such as inkjet printing, and which are stable.

Therefore, it is the object of the present invention to provide affordable, user-friendly sensors for detecting metabolites in biological samples obtained non-invasively.

It is another object of the present invention to provide methods of making the sensors.

It is yet another object of the present invention to provide methods of using the sensors.

### SUMMARY OF THE INVENTION

Inkjet-printed sensors for detecting metabolites in biological samples obtained non-invasively are provided. The devices typically include a backing layer, and at least one set of three electrodes. In a preferred embodiment, the electrodes are printed from a conducting polymer onto the backing layer. An exemplary device includes a three-electrode geometry which include a reference electrode, a working electrode, which preferably includes a biofunctional polymeric coating, and a counter electrode. Typically, each electrode include an active area, an electrical interconnect, and a contact area.

The electrodes may have a length between about 2 mm and about 20 mm, a width between about 0.1 mm and about 2 mm, and a height between about 0.1 mm and about 2 mm. The sensors may include an array of sets of three electrodes. The sensor may be connected to a data acquisition system, a display system, or both an acquisition and a display system, forming a sensor system.

Generally, the working electrode includes a biofunctional coating positioned over its active, and a biofunctional molecule in the biofunctional coating. The sensor typically includes a sensing area. The sensing area is usually formed of at least a portion of the active areas of the reference electrode, the working electrode, and the counter electrode. In some preferred embodiments, the sensing areas is formed of all of the active area of the reference electrode, all of the active areas of the working electrode and all of the active areas of the counter electrode. The sensing area may include a protective coating. The contact areas of the reference electrode, the working electrode, and the counter electrode connect the sensor to a data acquisition system, a display system, or both an acquisition and a display system, forming a sensor system. The electrical interconnects that connect the sensing area and the contact areas of the electrodes may include an insulation coating.

The electrodes of the sensor may be printed from a conducting polymer. Suitable conducting polymers include poly(4,4-dioctylcyclopentadithiophene), poly(isothianapthene), poly(3,4-ethylenedioxythiophene), polyacetylene (PAC), polyaniline (PANI), polypyrrole (PPY) or polythiophenes (PT), poly(p-phenylene sulfide) (PPS), and poly(3,4 ethylenedioxythiophene) polystyrene sulfonate (PEDOT:PSS). The sensing area may include a protective coating. Typically, the protective coating is a polymer that reduces or prevents the non-specific interaction or interference of different molecules in the biological sample with the biofunctional coating of the sensor. The protective coating can be a cation exchange membrane containing a polymer that prevent negatively charged interferences from reaching the sensor surface. Exemplary polymers that may be used as or in a protective coating include polystyrene sulfonate, perfluorinated sulfonated ionomer such as Nafion® (E. I. Du Pont De Nemours And Company Corporation, Wilmington, Del.), AQUIVION® (Solvay SA Corporation, Brussels, Belgium), or a combination thereof.

Typically, the biofunctional coating includes a mediator, such as a multivalent metal ion or an organometallic compound, and/or a polymer matrix formed of a positively charged polymer such as alginate amine, chitosan, dextran amine, heparin amine, and any combination thereof. The biofunctional coating also includes a biofunctional molecule, such as a carbohydrate, peptide, protein, or a nucleic acid, which is capable of oxidizing a biological molecule in a test sample. Exemplary biofunctional molecules include enzymes, co-factors, multivalent metal ions, and combination thereof. For example, the enzymes may be oxidases (Enzyme Commission Number (EC) 1.1.3) and/or oxido-reductases (EC 1.1.1, EC 1.1.5, EC 1.1.2, EC 1.1.6, EC 1.2.1, EC 1.4.1, EC 1.5.1, EC 1.6.1, EC 1.7.1, EC 1.8.1, EC 1.9.1, and EC 1.10.1). Other biofunctional molecules include molecules having the capability of acting as both electron donors and electron acceptors, e.g., multivalent metal ions such as copper, iron, magnesium, manganese, molybdenum, nickel and zinc, and co-factors such as nicotinamide adenine dinucleotide (NAD+), nicotinamide adenine dinucleotide phosphate (NADP+), ascorbic acid, flavin mononucleotide (FMN), flavin adenine dinucleotide (FAD), coenzyme F420, coenzyme B, Coenzyme Q, glutathione, heme, lipoamide, and pyrroloquinoline quinone.

The sensors may include either electrodes for amperometric tests or cyclovoltammetry, or organic electrochemical transistors for multiple detection of different metabolites with different enzymes combined with mediators and inkjet-printed on different substrates.

Generally, the sensor is small enough to be applied onto a medical device or onto a subject. The sensor's substrate (e.g., backing layer) may be a planar surface, such as a paper, a tattoo, a tape, a textile, a wound dressing or bandage, a medical implant, a contact lens, or a pad. The sensor may be part of a catheter, a contact lens, or a medical implant. The sensor may be worn by a subject on a patch or a bandage, or may be provided in a kit, ready to be used as needed. The sensor may be inserted in whole or in part into a biological sample such as blood, plasma, serum, urine, saliva, fecal matter, or cervicovaginal mucosa. The sensor may be connected to a data or signal acquisition system, such as a potentiostat, and, optionally, to a display system. The display system may be a portable display system with a screen to display sensor reading. Portable display systems include smartphones, tablets, laptops, desktop, pagers, watches, and glasses.

Typically, the sensor permits non-invasive detection of a presence, absence, or a concentration of, a biological molecule in a biological sample. Exemplary biological samples include bodily fluids or mucus, such as saliva, sputum, tear, sweat, urine, exudate, blood, plasma, or vaginal discharge. Exemplary biological molecules that may be detected with the sensor include biomarkers and metabolites, such as glucose, cholesterol, nicotine, carbon monoxide, nitrite, nitrate, alcohol, and bacterial metabolites.

### BRIEF DESCRIPTION OF THE DRAWINGS

FIGS. 1A, 1B, and 1C are diagrams showing the electrode inkjet-printed on paper. FIG. 1A shows a plurality of electrodes inkjet-printed on paper, forming a plurality of metabolite sensors 100. FIG. 1B is an enlarged diagram of the boxed region in FIG. 1A. Each sensor 100 includes a backing 12, a reference electrode 10, a working electrode 20, and a counter electrode 30. Each printed electrode includes an active area 82, an electrical interconnect 84, and a contact area 86. The combination of the active areas of the working, reference, and counter electrodes form the sensing area 40. Each printed electrode includes two layers of PEDOT:PSS. FIG. 1C is a diagram of the working electrode with all layers (electrode 50, dielectric 52, enzyme 54 and mediator 56, and Nafion® 58) printed successively. FIG. 1D is a scheme of the mechanism of the redox reaction. FIG. 1E is a scheme of the fully printed biosensor including dielectric layer 52, enzyme 54, mediator 56, Nafion® 58, and contact pads 70.

FIGS. 2A, 2B, and 2C are line graphs showing the cyclic voltammetry (20 mV/s) of a sensor. In FIG. 2A, the curve 1 represents the electrochemical reaction in PBS only, curve 2 represents the addition of 1 mM of glucose in the media, curve 3 is after the addition of 10 mM of glucose in the media, and curve 4 is before the deposition of the biological layer. FIGS. 2B and 2C show cyclic voltammetry (20 mV/s) of a printed sensor without a protective Nafion® coating (FIG. 2B), and with Nafion® printed over the electrode (2 layers, FIG. 2C). Curve 1 represents the electrochemical reaction is in PBS only, curve 2 represents the electrochemical reaction after the addition of 2 mM of lactate in the media, curve 3 after the addition of 0.01 mM of ascorbic acid in the media; curve 4 is after the addition of 0.15 mM of uric acid in the media, and curve 5 is after the addition of 1 mM of glucose in the media.

FIGS. 3A, 3B, 3C, 3D and 3E are graphs showing the amperometric measurements (applied potential of 0.25V) for different concentrations of glucose added successively in PBS (FIG. 3A), the normalized response of the sensor to the addition of glucose in PBS with Nafion® (curve 1) and without Nafion® (curve 2), n=3 (FIG. 3B), the normalized response of the sensor to the addition of glucose in PBS with Nafion® (curve 1) and without Nafion® (curve 2) presented on the linear scale for y axis (FIG. 3C), and cyclic voltammetry (20 mV/s) of a printed sensor, repeated 10 times in presence of glucose (1 mM) n=3 (FIG. 3D). For the data in FIG. 3D, after each addition of glucose, the sensor was rinsed with PBS and CV was recorded in media only to verify the reusability of the device and then 1 mM of glucose solution was added in the media and CVs were recorded (solid lines). FIG. 3E is a graph showing the normalized response of the device measured 24h after the introduction of glucose in the media (1 mM) the experiments were carried for a total duration of 1 month.

FIGS. 4A, 4B, and 4C are graphs showing the amperometric measurements (applied potential of 0.25V) for different concentrations of glucose added successively in saliva (FIG. 4A), the normalized response of the sensor to the addition of glucose in saliva with Nafion® n=3 (FIG. 4B), and the normalized response of the sensor to the addition of glucose in saliva with Nafion® presented on the linear scale for y axis (FIG. 4C).

FIGS. 5A and 5B are graphs showing the cyclic voltammetry (20 mV/s) of a printed sensor in PBS only (curve 1) in presence of glucose, 1 mM (curve 2) and 10 mM (curve 3). In both configurations the working electrode is PEDOT:PSS. In FIG. 5A, the reference and counter electrodes are Agl/AgCl electrodes, in FIG. 5B the reference and counter electrodes are PEDOT:PSS electrodes.

FIGS. 6A and 6B are graphs showing the cyclic voltammetry (20 mV/s) of a printed biosensor (both working, counter and reference electrodes are composed of PEDOT:PSS) in PBS only (FIG. 6A) and in presence of glucose (1 mM) (FIG. 6B). The curve 0 is representing the working electrode with PEDOT:PSS only. The curves 2, 4, and 6 are respectively 2, 4, and 6 layers of printed enzyme with mediator.

FIG. 7 is a graph showing the amperometric measurements (applied potential of 0.25V) for different concentrations of glucose added successively in PBS without Nafion®. To decrease the concentration of glucose, glucose is extracted from the system and PBS is added accordingly.

FIGS. 8A and 8B are graphs showing the cyclic voltammetry (20 mV/s) of a printed biosensor (both working, counter and reference electrodes are composed of PEDOT:PSS) in saliva with increasing number of printed Nafion® layers (0,1,2,4) (FIG. 8A), and the comparison of the CVs with 2 layers of Nafion® printed in PBS (curve 1) and in saliva (curve 2) and in saliva after addition of 1 mM and 10 mM of glucose to the system (curves 3 and 4) (FIG. 8B).

### DETAILED DESCRIPTION OF THE INVENTION

### I. Definitions

As used herein, the term “sensor” refers to a device containing elements required for generating an electrical current when a biological sample is applied to the sensor. The sensor may include additional elements, such as an acquisition system and/or a display system, forming a sensor system.

As used herein, the term “metabolite” refers to a small molecule formed during or after a metabolic reaction, or a metabolic pathway.

As used herein, the term “detection” or “detecting” in the context of detecting a metabolite using a sensor, refers to an act of obtaining a value or a reading indicating the presence or absence of the metabolite in a sample. The detection may require a comparison of the obtained value or reading for a given metabolite from a test sample to a value or reading obtained from a control sample for the same metabolite and tested in the same way as the test sample.

As used herein, the term “mediator” as refers to a molecule capable of participating in an electron exchange between the metabolite, the biofunctional molecule, and/or the conducting polymer.

As used herein, the term “biofunctional” in the context of a molecule or a coating refers to a property of the molecule or the coating capable of electron exchange.

As used herein, the term “planar surface” refers to a surface with a region that is sufficiently planar, i.e., sufficiently flat, over a surface area sufficient to accommodate an electrode. For example, if a planar surface is a contact lens, the contact lens has a sufficiently planar region to accommodate an electrode having a length of about 2 mm, and a width of about 2 mm.

As used herein, the term “ink” refers to a solution or suspension of a material to be deposited using inkjet printing onto a surface, such as a conducting polymer or metal, or a polymeric coating

As used herein, the term “measuring,” in the context of the disclosed method, refers to one or more steps taken to detect a level, an intensity (such as a normalized intensity), an amount, or a concentration, for a given substance, molecule or compound such as a metabolite or an enzyme.

As used herein, the term “biomarker” or “marker” refers to a substance, molecule, or compound that is produced by, synthesized, secreted, or derived, at least in part, from the subject and is used to determine presence or absence of a disease, and/or the severity of the disease.

As used herein, the term “stability” refers to the sensor's capability to preserve at least about 80% of its original signal.

As used herein, the term “fold” refers to a difference in the number of times. For example, “1.5 fold greater than” refers to a value that is 1.5 as large as a given reference value. Fold values can also be expressed in percentage. For example, 1.5 fold is equivalent to 150% of the reference value.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%.

### II. Sensor System

Printed enzymatic sensors and sensor systems capable of detecting metabolite concentrations in the relevant range from biological samples obtained non-invasively show long term stability use with accurate and reproducible measurement of the metabolite.

The sensor system typically includes a sensor, which may be attached to a reader containing an acquisition and/or a display component. The sensor system is portable, and the acquisition and/or a display components may be attached or disconnected from the sensor as needed.

A. Sensor

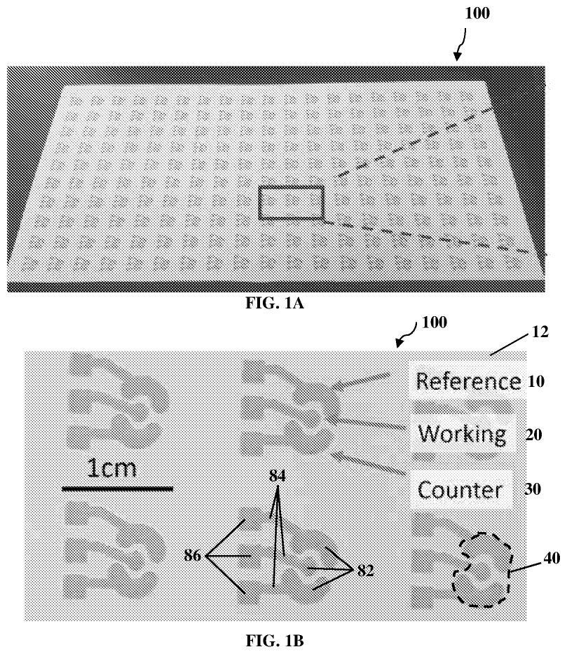

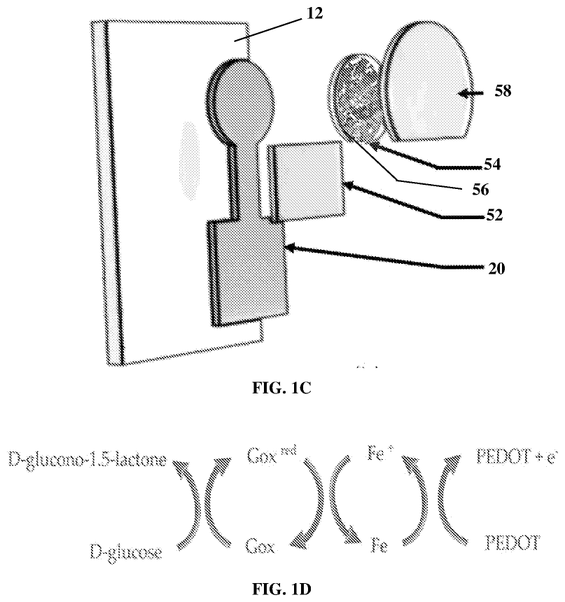

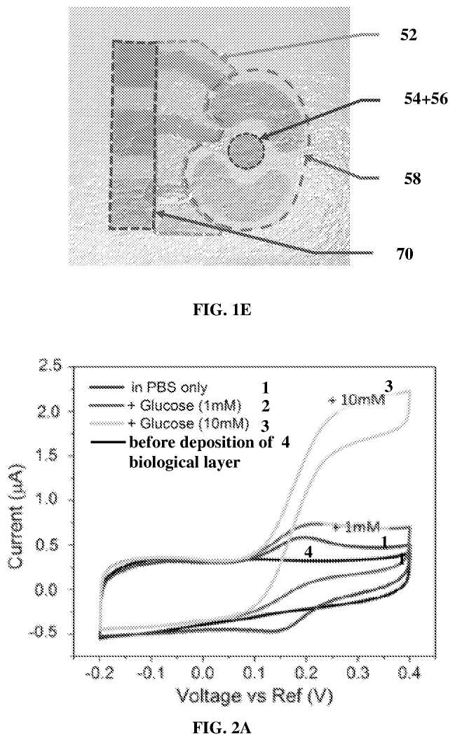

The sensors typically include at least one backing layer, and at least one set of three electrodes printed from a conducting material onto the backing layer. Typically, the electrodes include an active area, an electrical interconnect, and a contact area. The electrodes can be formed from the same conducting material or different conducting materials. Typically, all electrodes are formed from the same conducting material, i.e. a conducting polymer. In some instances, all electrodes can be printed from the same conducting polymer on the backing layer in one step. FIGS. 1A-1C are diagrams showing one of the embodiments of a sensor. An exemplary sensor 100 includes a backing 12, a reference electrode 10, a working electrode 20, and a counter electrode 30. Each electrode includes an active area 82, an electrical interconnect 84, and a contact area 86. The combination of the active areas of the reference electrode 10, the working electrode 20, and the counter electrode 30 forms the sensing area 40. Each printed electrode may include 1, 2, 3, 4, 5, 6, 7, 8, 9, or 10 layers of a conducting polymer such as poly(3,4-ethylenedioxythiophene) doped with poly(styrene sulfonate) anions (PEDOT/PSS), which is widely used in various organic optoelectronic devices. PEDOT: PSS is a blend of cationic polythiopene derivative, doped with a polyanion. FIG. 1C is a diagram of the working electrode with all layers (electrode 20, dielectric 52, enzyme 54 and mediator 56, and Nafion® 58) printed successively.

An exemplary set includes a three electrode geometry with a reference electrode, a working electrode with a biofunctional coating, and a counter electrode. Typically, the electrodes have a length between about 2 mm and about 20 mm, a width between about 0.1 mm and about 2 mm, and a height between about 0.1 mm and about 2 mm.

The sensors may include an array of sets of three electrodes. The sensor may be connected to an acquisition system, a display system, or both an acquisition and a display system. The contact areas of the reference electrode, the working electrode, and the counter electrode may connect the sensor to a data acquisition system, a display system, or both an acquisition and a display system, forming a sensor system.

Generally, the sensors include a biofunctional coating positioned over a surface of the working electrode, i.e. the active area of the working electrode, and an electron-generating biofunctional molecule in the biofunctional coating. The biofunctional coating may further include a mediator and/or a polymer matrix. The sensor may include a sensing area, which is formed of active areas of the reference electrode, the working electrode, and the counter electrode. The sensing area typically includes a protective coating. Typically, the protective coating is a polymer that reduces or prevents the non-specific interaction or interference of different molecules in the biological sample with the biofunctional coating of the sensor. The protective coating may also stabilize the biofunctional molecules and/or the mediators in the biofunctional coating. The protective coating can be a cation exchange membrane containing a polymer that prevent negatively charged interferences from reaching the sensor surface. The electrical interconnects that connect the sensing area and the contact areas of the electrodes may include an insulation coating, such as a dielectric coating. The dielectric coating can separate or insulate the sensing area from the contact areas.

The sensor system can include a printed metabolite sensor on a backing layer, which can be as simple as a commercial disposable paper. As the Examples demonstrate, an exemplary sensor can be made by combining biocompatible conducting polymer poly(3,4 ethylenedioxythiophene) polystyrene sulfonate (PEDOT:PSS) as the transducer, dielectric and biological inks towards the realization of highly sensitive, selective, portable, inexpensive, stable, and user-friendly enzymatic sensing device. The printed sensor was tested over a period of one month and its long-term stability was confirmed. This demonstrated that the sensor may be used in real world applications with bodily fluids such as blood and saliva, enabling non-invasive monitoring. The sensor may be all-polymer “smart e-paper biosensor” providing the next generation of disposable low cost and eco-friendly high-performance biomedical devices.

1. Reference Electrode

The reference electrode is an electrode having a maintained potential, used as a reference for measurement of other electrodes. Exemplary reference electrodes are, but not limited to, silver, silver chloride, silver/silver chloride, gold, copper, carbon, and conducting polymer. The reference electrode may be screen-printed or inkjet-printed from the above-mentioned materials. Typically, the reference electrode is inkjet-printed from a conducting polymer.

2. Working Electrode

The working electrode typically includes a biofunctional coating. The biofunctional coating may contain a biofunctional molecule. The biofunctional coating may further include a mediator and/or a polymer matrix.

The mechanism of the detection of the metabolite is based on a cycle of electrochemical reactions, which alternatively oxidize/reduce the compounds immobilized at the surface of the sensor, i.e. at the surface of the working electrode. Typically, the electrons are transferred from the biological molecule to the conducting polymer through the cycle of electrochemical reactions, generating a current between the working and counter electrodes detected by the acquisition system. An exemplary cycle of reactions is depicted in FIG. 1D, where upon reacting with a biological molecule, i.e. glucose, the biofunctional molecule, i.e. GOx gets reduced, and the reduced biofunctional molecule cycles back via the mediator, i.e. Fc, which mediated electron transfer from the biofunctional molecule to the conducting polymer, i.e. PEDOT:PSS.

a. Biofunctional Molecules

The biofunctional coating includes a biofunctional molecule, such as a carbohydrate, peptide, protein, or a nucleic acid, which is capable of oxidizing or reducing a biological molecule in a test sample. In some instances, the biofunctional molecule is capable of oxidizing a biological molecule in a test sample. In some instances, the biofunctional molecule is capable of reducing a biological molecule in a test sample. Exemplary biofunctional molecules include enzymes, enzymes with co-factors, multivalent metal ions, and any combination thereof. For example, the enzymes may be oxidases (Enzyme Commission Number (EC) 1.1.3) and/or oxido-reductases (EC 1.1.1, EC 1.1.5, EC 1.1.2, EC 1.1.6, EC 1.2.1, EC 1.4.1, EC 1.5.1, EC 1.6.1, EC 1.7.1, EC 1.8.1, EC 1.9.1, and EC 1.10.1).

i. Oxidases

Exemplary oxidase enzymes that may be used in the sensors include Glucose oxidase (Enzyme Commission Number (EC) 1.1.3.4); Lactate oxidase (EC 1.1.3.2); Hexose oxidase (EC 1.1.3.5), Cholesterol oxidase (EC 1.1.3.6), Aryl-alcohol oxidase (EC 1.1.3.7), L-gulonolactone oxidase (EC 1.1.3.8), Galactose oxidase (EC 1.1.3.9), Pyranose oxidase (EC 1.1.3.10), L-sorbose oxidase (EC 1.1.3.11), Pyridoxine 4-oxidase (EC 1.1.3.12), Alcohol oxidase (EC 1.1.3.13), Catechol oxidase (dimerizing) (EC 1.1.3.14), (S)-2-hydroxy-acid oxidase (EC 1.1.3.15), Ecdysone oxidase (EC 1.1.3.16), Choline oxidase (EC 1.1.3.17), Secondary-alcohol oxidase (EC 1.1.3.18), 4-hydroxymandelate oxidase (decarboxylating) (EC 1.1.3.19), Long-chain-alcohol oxidase (EC 1.1.3.20), Glycerol-3-phosphate oxidase (EC 1.1.3.21), Thiamine oxidase (EC 1.1.3.23), hydroxyphytanate oxidase (EC 1.1.3.27), Nucleoside oxidase (EC 1.1.3.28), N-acylhexosamine oxidase (EC 1.1.3.29), Polyvinyl-alcohol oxidase (EC 1.1.3.30), D-arabinono-1,4-lactone oxidase (EC 1.1.3.37), Vanillyl-alcohol oxidase (EC 1.1.3.38), Nucleoside oxidase (H(2)O(2)-forming) (EC 1.1.3.39), D-mannitol oxidase (EC 1.1.3.40), Alditol oxidase (EC 1.1.3.41), Prosolanapyrone-II oxidase (EC 1.1.3.42), Paromamine 6′-oxidase (EC 1.1.3.43), 6′″-hydroxyneomycin C oxidase (EC 1.1.3.44), Aclacinomycin-N oxidase (EC 1.1.3.45), 4-hydroxymandelate oxidase (EC 1.1.3.46), 5-(hydroxymethyl)furfural oxidase (EC 1.1.3.47), 3-deoxy-alpha-D-manno-octulosonate 8-oxidase (EC 1.1.3.48), and (R)-mandelonitrile oxidase (EC 1.1.3.49).

ii. Oxido-Reductases

Exemplary oxido-reductase enzymes that may be used in the sensors include (R,R)-butanediol dehydrogenase (EC 1.1.1.4), D-Xtkykise redyctase (EC 1.1.1.9), I-Xylulose reductase (EC 1.1.1.10), Glucuronate reductase (EC 1.1.1.19), Aldehyde reductase (EC 1.1.1.21), Quinate dehydrogenase (EC 1.1.1.24), Mevaldate reductase (EC 1.1.1.32), Hydroxymethylglutaryl-CoA reductase (EC 1.1.1.34), Fructuronate reductase (EC 1.1.1.57), 17-beta-estradiol 17-dehydrogenase (EC 1.1.1.62), Lactaldehyde reductase (EC 1.1.1.77), Glyoxylate reductase (EC 1.1.1.79), Hydroxypyruvate reductase (EC 1.1.1.81), Homoisocitrate dehydrogenase (1.1.1.87), Glycerol-3-phosphate dehydrogenase (NAD(P)(+)) (EC 1.1.1.94), Phosphoglycerate dehydrogenase (EC 1.1.1.95), 3-Oxoacyl-[acyl-carrier-protein] reductase (EC 1.1.1.100), 3-dehydrosphinganine reductase (EC 1.1.1.102), UDP-N-acetylglucosamine 6-dehydrogenase (EC 1.1.1.136), 2-Dehydropantoate 2-reductase (EC 1.1.1.169), Cholest-5-ene-3-beta,7-alpha-diol 3-beta-dehydrogenase (EC 1.1.1.181), Long-chain-alcohol dehydrogenase (EC 1.1.1.192), 1,3-propanediol dehydrogenase (EC 1.1.1.202), 3-beta-hydroxy-5-beta-steroid dehydrogenase (EC 1.1.1.277), 8-hydroxygeraniol dehydrogenase (EC 1.1.1.324), Nicotine blue oxidoreductase (EC 1.1.1.328), dTDP-3,4-didehydro-2,6-dideoxy-alpha-D-glucose 3-reductase (EC 1.1.1.384), L-lactate dehydrogenase (cytochrome)(EC 1.1.2.3), D-lactate dehydrogenase (cytochrome) (EC 1.1.2.4), Quinoprotein glucose dehydrogenase (PQQ, quinone) (EC 1.1.5.2), Malate dehydrogenase (quinone) (EC 1.1.5.4), F420H(2):quinone oxidoreductase (EC 1.1.98.4), L-2-hydroxyglutarate dehydrogenase (EC 1.1.99.2), Glycolate dehydrogenase (EC 1.1.99.14), Glucose-fructose oxidoreductase (EC 1.1.99.28), Pyruvate dehydrogenase (NADP(+)) (EC 1.2.1.51), Carbon-monoxide dehydrogenase (cytochrome b-561) (EC 1.2.2.4), Pyruvate dehydrogenase (acetyl-transferring) (EC 1.2.4.1), Oxoglutarate dehydrogenase (succinyl-transferring) (EC 1.2.4.2), 3-methyl-2-oxobutanoate dehydrogenase (2-methylpropanoyl-transferring) (EC 1.2.4.4), pyruvate synthase (EC 1.2.7.1), 2-oxoglutarate synthase (EC 1.2.7.3), Aldehyde ferredoxin oxidoreductase., EC 1.2.7.5; Glyceraldehyde-3-phosphate dehydrogenase (ferredoxin), EC 1.2.7.6; 3-methyl-2-oxobutanoate dehydrogenase (ferredoxin), EC 1.2.7.7; Indolepyruvate ferredoxin oxidoreductase, EC 1.2.7.8; Oxalate oxidoreductase, EC 1.2.7.10; 2-oxoacid oxidoreductase (ferredoxin), EC 1.2.7.11; Aldehyde dehydrogenase (FAD-independent), EC 1.2.99.7; Glyceraldehyde dehydrogenase (FAD-containing), EC 1.2.99.8; Enoyl-[acyl-carrier-protein] reductase (NADH) (EC 1.3.1.9), Enoyl-[acyl-carrier-protein] reductase (NADPH, B-specific) (EC 1.3.1.10), Orotate reductase (NADH) (EC 1.3.1.14), Dihydrodipicolinate reductase (EC 1.3.1.26), Isoquinoline 1-oxidoreductase, EC 1.3.99.16; Quinoline 2-oxidoreductase, EC 1.3.99.17; Quinoline-4-carboxylate 2-oxidoreductase, EC 1.3.99.19; All-trans-retinol 13,14-reductase, EC 1.3.99.23; Serine 2-dehydrogenase, EC 1.4.1.7; Glutamate synthase (NADPH), EC 1.4.1.13; Methylenetetrahydrofolate reductase (NAD(P)H), Pyrroline-5-carboxylate reductase (EC 1.5.1.2), Dihydrofolate reductase (EC 1.5.1.3), Methylenetetrahydrofolate reductase (NADP+) (EC 1.5.1.5), EC 1.5.1.20; Flavin reductase (NADPH), EC 1.5.1.30; 6,7-dihydropteridine reductase, EC 1.5.1.34; 8-hydroxy-5-deazaflavin:NADPH oxidoreductase, EC 1.5.1.40; Dihydromethanopterin reductase (NAD(P)(+)). EC 1.5.1.47; alkylglycine oxidase, EC 1.5.3.20; Glyphosate oxidoreductase, EC 1.5.3.23; Electron-transferring-flavoprotein dehydrogenase, EC 1.5.5.1; Coenzyme F420 oxidoreductase (ferredoxin), EC 1.5.7.2; 5,10-methylenetetra-hydromethanopterin reductase, EC 1.5.98.2; NAD(P)(+) transhydrogenase, EC 1.6.1.3; NADPH-hemoprotein reductase, EC 1.6.2.4; Cystine reductase (NADH) (EC 1.6.4.1), NAD(P)H dehydrogenase (quinone), EC 1.6.5.2; NADH:ubiquinone reductase (H(+)-translocating). EC 1.6.5.3; NADPH:quinone reductase, EC 1.6.5.5; NADH dehydrogenase, Nitrate reductase (NADH) (EC 1.6.6.1), Nitrate reductase [NAD(P)H] (EC 1.6.6.2), Nitrate reductase (NADPH) (EC 1.6.6.3), Nitrite reductase [NAD(P)H] (EC 1.6.6.4), GMP reductase (EC 1.6.6.8), EC 1.6.99.3; Nitrate reductase (NADH). EC 1.7.1.1; Nitrate reductase (NADPH), EC 1.7.1.3; Nitrite reductase (NAD(P)H), EC 1.7.1.4; Hyponitrite reductase, EC 1.7.1.5; Nitrite reductase (NADH), EC 1.7.1.15; Nitrite reductase (NO-forming), EC 1.7.2.1; Hydroxylamine oxidase (cytochrome), EC 1.7.3.6; Nitrate reductase (quinone), EC 1.7.5.1; Ferredoxin-nitrate reductase (EC 1.7.7.1), Nitrate reductase (EC 1.7.99.4), Dihydrolipoyl dehydrogenase, EC 1.8.1.4; 2-oxopropyl-CoM reductase (carboxylating), EC 1.8.1.5; Cystine reductase, EC 1.8.1.6; Glutathione-disulfide reductase, EC 1.8.1.7; Thioredoxin-disulfide reductase. EC 1.8.1.9; CoA-glutathione reductase, EC 1.8.1.10; Sulfite reductase (ferredoxin) (EC 1.8.7.1), Sulfite reductase (EC 1.8.99.1), Adenylsulphate reductase (EC 1.8.99.2),1-Ascorbate-cytochrome-b5 reductase (EC 1.10.2.1), Ubiquinol-cytochrome-c reductase (EC 1.10.2.2), Plastoquinol-plastocyanin reductase (EC 1.10.99.1), and Ribonucleoside-diphosphate reductase (EC 1.17.4.1).

iii. Biological Molecules

Biological molecules that may be oxidized or reduced by the biofunctional molecules, and thus detected by the sensor include biomarkers and metabolites, such as glucose, cholesterol, nicotine, carbon monoxide, nitrite, nitrate, alcohol, and bacterial metabolites. Monitoring metabolite levels (such as glucose) can provide very useful information regarding key metabolic activities in the body and detect associated irregularities such as in the case of diabetes, a worldwide chronic disease which affects nearly 1 in 11 of the world's adult population.

Metabolites detected by the sensors include metabolites of energy metabolism, carbohydrate and lipid metabolism, nucleotide and amino acid metabolism in a biological sample obtained from a subject.

For example, the metabolites may be metabolites of any one of the following biochemical pathways: carbohydrate and lipid metabolism, including central carbohydrate metabolism, fatty acid metabolism, lipid metabolism, lipopolysaccharide metabolism, glycan metabolism, glycosaminoglycan metabolism, sterol biosynthesis; nucleotide and amino acid metabolism, including purine metabolism, pyrimidine metabolism, serine and threonine metabolism, cysteine and methionine metabolism, branched-chain amino acid metabolism, branched-chain amino acid metabolism, lysine metabolism, histidine metabolism, aromatic amino acid metabolism, other amino acid metabolism, cofactor and vitamin biosynthesis, polyamine biosynthesis; and secondary metabolism, including aromatics degradation, and biosynthesis of secondary metabolites.

The metabolites may be glucose, cholesterol, nicotine, carbon monoxide, nitrite, nitrate, alcohol, bacterial metabolites, pyruvate, oxaloacetate, fructose-6-phosphate, acetyl coenzyme A (acetyl-CoA), oxoglutarate, 2-oxoglutarate, pentose phosphate, glucose 6-phosphate, ribulose 5-phosphate, ribose 5-phosphate, phosphoribosyl pyrophosphate, glyceraldehyde-3-phosphate, gluconate, glycerate-3-phosphate, Glycerol-3-phosphate, gluconate, galactonate, glycerate, propanoyl coenzyme A (propanoyl-CoA), galactose, alpha-D-glucose-1-phosphate, D-galactonate, D-glucose 1-phosphate, glutamine, methionine, valine, hypoxanthine, inosine, isoleucine, sphingosine, palmitoylcarnitine, lysoPC(18:2), C8-ceramide, linoleamide, lysoPC(22:5), lysoPC(20:3), palmitic amide, uric acid, choline, creatine, L-glutamine, alanine, creatinine, and N-acetyl-L-aspartate, tyrosinamide, biotin sulfone, hexanoic acid, 1-aminonaphthalene, 7-dehydrocholesterol, azelaic acid, acetone, 3-hydroxybutyrate, 1-methylhistamine, 1-methylnicotinamide, 2-methylglutarate, 2-oxoglutarate, 3-OH-3-methylglutarate, 3-methyladipate, 4-aminohippurate, acetone, adenine, alanine, creatine, dimethylamine, formate, fumarate, glucose, glycolate, imidazole, lactate, methylamine, O-acetylcarnitine, oxalacetate, phenylacetylglycine, phenylalanine, tryptophan, tyrosine, cis-aconitate, myo-inositol, trans-aconitate, leucine, valine, acetate, acetoacetate, creatinine, and trimethylalanine-N-oxide, gamma-aminobutyric acid (GABA), uric acid, citric acid, hypoxanthine, and inosine.

b. Mediators

Typically, a mediator is a small molecule compound participating in an electron donor/acceptance. Exemplary mediators include compounds containing multivalent metal ions such as copper, iron, magnesium, manganese, molybdenum, nickel and zinc, organometallic compounds, phenazine methosulfate, dichlorophenol indophenol, short chain ubiquinones, ferrocene complex, and co-factors such as nicotinamide adenine dinucleotide (NAD+), nicotinamide adenine dinucleotide phosphate (NADP+), ascorbic acid, flavin mononucleotide (FMN), flavin adenine dinucleotide (FAD), coenzyme F420, coenzyme B, Coenzyme Q, glutathione, heme, lipoamide, and pyrroloquinoline quinone. In some instances, the mediator is a ferrocene complex.

3. Counter Electrode

The counter electrode, often also called the auxiliary electrode, is an electrode used in a three electrode electrochemical cell for voltammetric analysis or other reactions in which an electric current is expected to flow. Exemplary counter electrodes are, but not limited to, gold, copper, carbon, and conducting polymer. The counter electrode may be screen-printed or inkjet-printed from the above-mentioned materials. Typically, the counter electrode is inkjet-printed from a conducting polymer.

4. Materials Forming the Electrodes

Generally, the sensor includes at least one set of three electrodes. Each electrode in the sensor may include one or more coatings. The electrode and the coatings can be inkjet-printed, in sequential manner, to obtain the arrangement described in section Sensors.

Materials forming the electrodes and its coatings include conductive polymers, dielectric inks, charged biocompatible polymers, and synthetic ionic polymers. Typically, the reference electrode, the working electrode, and the counter electrode are formed of conductive polymers. The reference electrode, the working electrode, and the counter electrode may also include a dielectric coating formed of dielectric ink. The working electrode typically includes a biofunctional coating containing a biofunctional molecule, a mediator, and polymer matrix formed of a charged biocompatible polymer. At least a portion, i.e., the active area of the reference electrode, the working electrode, and the counter electrode may be coated with a protective coating containing a synthetic ionic polymer.

a. Conducting Polymer

Conducting polymers which can be used to form the reference electrode, the working electrode, and the counter electrode. Exemplary conducting polymers include poly(3,4-ethylenedioxythiphene) (PEDOT), poly(hydrooxymethyl 3,4-ethylenedioxythiphene) (PEDOT-OH), polystyrenesulfonate (PSS), F8BT, F8T2, J51, MDMO-PPV, MEH-PPV, PBDB-T, PBDTBO-TPD, PBDT(EH)-TPD, PBDTTT-C-T, PBDTTT-CF, PBTTPD, PBTTT-C14, PCDTBT, PCPDTBT, PDTSTPD, PffBT4T-20D, PfifiT4T-C9C13, PFO-DBT, Poly([2,6′-4,8-di(5-ethylhexylthienyl)benzo[1,2-b;3,3-b]dithiophene] {3-fluoro-2 [(2-ethylhexyl)carbonyl]thieno[3,4-b]thiophenediyl}), Poly(3-dodecylthiophene-2,5-diyl), Poly(3-hexylthiophene-2,5-diyl), Poly(3-octylthiophene-2,5-diyl), PSiF-DBT, poly(triaryl amine) (PTAA), PTB7, TQl, N2300, P(NDI-T2), poly(diketopyrrolopyrrole) (DPP), poly(benzimidazobenzophenanthroline), poly(2,5-di(3,7-dimethyloctyloxy)cyanoterephthalylidene), poly(2,5-di(hexyloxy)cyanoterephthalylidene), poly(5-(3,7-dimethyloctyloxy)-2-methoxy-cyanoterephthalylidene), poly(2,5-di(octyloxy)cyanoterephthalylidene), poly(5-(2-ethylhexyloxy)-2-methoxy-cyanoterephthalylidene), poly(4,4-dioctylcyclopentadithiophene), poly(isothianapthene), poly(3,4-ethylenedioxythiophene), polyacetylene (PAC), polyaniline (PANI), polypyrrole (PPY) or polythiophenes (PT), and poly(p-phenylene sulfide) (PPS). In some instances, the conductive polymer is a combination of two or more conductive polymers described above. For example, the conductive polymer can be poly(3,4 ethylenedioxythiophene):polystyrene sulfonate (PEDOT:PSS).

b. Dielectric Coating

The dielectric coating may be a dielectric/insulator ink layer. The dielectric ink layer may be a dielectric polymer, copolymer, block polymer, or polymer-inorganic composite. The dielectric polymer may be polyimide, polyurethane, polysiloxane, polyacrylate, plyethylene, polystyrene, polyepoxide, polytetrafluoroethylene, polyarelene ether, methylsilsesquioxone, fluorinated polyimide, or a combination thereof. Dielectric polymer-inorganic composite may include a polymer and an inorganic compound such as BaTiO₃, TiO₂, Al₂O₃, ZrO₂. Exemplary dielectric polymer-inorganic composite may be polyimide-BaTiO₃. Commercially available dielectric/insulator inks or pastes may be EMD 6200 (Sun Chemical Corporation, Parsippany, N.J.), KA 701 (DuPont), 125-17, 116-20, 113-48, 111-27, 118-02, 122-01, 119-07, 118-08, 118-12 (CREATIVE MATERIALS®), D2070423P5, D2071120P1, D2140114D5, D2020823P2, D50706P3, D2030210D1, D2070412P3, D2081009D6, D50706D2, D2130510D2 (Sun Chemical Corporation, Parsippany, N.J.), LOCTITE® EDAG 1020A E&C, LOCTITE® EDAG 452SS E&C, LOCTITE® EDAG PD 038 E&C, LOCTITE® EDAG PF 021 E&C, LOCTITE® EDAG PF 455B E&C, or LOCTITE® M 7000 A BLU E&C (Henkel Corporation).

c. Polymer Matrices Immobilizing

Biofunctional Molecules

In some instances, the biofunctional coating of the working electrode includes a mediator, a biofunctional molecule, and a polymer matrix for immobilizing the mediator and the biofunctional molecule. The polymer matrix can entrap the mediator and the biofunctional molecules within its matrix to prevent leaking and to improve the processability of the biofunctional molecules. The polymer matrix can be biocompatible.

The polymer matrices for immobilizing the mediator and the biofunctional molecule may be formed of positively charged polymers, such as alginate amine, chitosan, dextran amine, heparin amine, and any combination thereof.

d. Protective Coating

The protective coating is typically inkjet-printed over the electrodes, over a portion of the electrodes, and may be the outermost-layer of on the electrodes. The protective coating may be formed of synthetic ionic polymer, such as polystyrene sulfonate and perfluorinated sulfonated ionomers, such as Nation®, AQUIVION® (Solvay Sa Corporation, Brussels Belgium), or a combination thereof.

5. Sensing Area

The sensing area typically includes a portion of the working, counter and reference electrodes, i.e. the active areas of the working, counter, and reference electrodes (FIG. 1B). Typically, the active area of the working electrode containing at least a portion of the biofunctional coating. The sensing area may include a polymer coating. The polymer coating typically reduces or prevents the non-specific interaction or interference of different molecules in the biological sample with the biofunctional molecule of the sensor. The polymer coating reduces or prevents any interaction or interference with the electron transport in the sensor from the different molecules in the biological sample.

6. Backing Layer or Substrate

The sensor's backing layer may be a planar surface such as paper, a tattoo, a tape, a textile, a wound dressing or bandage, a medical implant such as catheter, a contact lens, a patch, a pad, glass, or plastics. Typically, the backing layer is a paper. The paper may be disposable after one use or multiple uses, i.e. four times.

B. Reader

The sensors may be connected to a system, optionally including a display.

a. Acquisition System

An acquisition system may be a potentiostat, a biosensor, or a galvanostat. Typically, the acquisition system is connected to software that converts data into a graph, chart or table, for a compound or molecule such as a metabolite.

b. Display System

The display system may be a portable display system with a screen to display sensor reading. Portable display systems include smartphones, tablets, laptops, and monitors.

C. Packaging

The sensor may be packaged to protect the electrodes prior to use. Examples of packaging are known in the art and include molded or sealed pouches with temperature and/or humidity control. The pouches may be foil pouches, paper pouches, cardboard boxes, polymeric pouches, or a combination thereof.

The sensors and sensor systems may be packaged as one unit. Alternatively, the sensors may be packaged separately, and used as needed with an acquisition and/or display system provided by the end user.

### III. Methods of Making the Sensors and the Sensor Systems

Inkjet technology may be used in all the steps for the fabrication of a noninvasive metabolite sensing device. Conducting polymers have attracted a great deal attention due to their unique set of features such as their combined ionic and electronic conduction, their soft nature and ease in processability rendering them an ideal alternative to the inorganic materials used to date for biosensing applications. The use of conducting polymers such as poly(3,4-ethylenedioxythiophene) polystyrene sulfonate (PEDOT:PSS), and additive printing technologies, such as screen printing or inkjet printing, yields high performance biomedical devices.

For the deposition of the electronic components as well as the biological layers (such as enzymes for the enzymatic based metabolite sensors), inkjet technology not only allows for the controlled deposition of a variety of different materials but also constitutes a low temperature process which is a critical factor when it comes to the integration of biological molecules such as enzymes. Inkjetting enables the patterning of customizable geometries and can easily be integrated in roll-to-roll processes.

A general method of making the sensors include using a conducting polymer ink dedicated for inkjetting and adjusting the ink formulation to meet the substrate requirements for the formation of a uniform and conducting layer. For example, a cross linker, i.e. 3-glycidoxypropyltrimethoxysilane (GOPS) and/or a surfactant, i.e. dodecyl benzene sulfonic acid (DBSA) may be added to the conducting polymer ink to prevent delamination of the conducting pattern from the backing layer and to improve the wettability of the ink and film formation during printing, respectively. The cross linker can be added at a concentration between about 0.01 wt % and about 5 wt %, between about 0.1 wt % and about 5 wt %, between about 0.5 wt % and about 5 wt %, between about 0.5 wt % and about 4 wt %, between about 0.5 wt % and about 2 wt %, between about 1 wt % and about 5 wt %, and between about 0.1 wt % and about 1 wt %. In some instances, the cross linker can be added at a concentration of about 1 wt %. In some instances, the cross linker is absent. The surfactant can be added at a concentration of between about 0.01% (v/v) and about 1% (v/v), between about 0.05% (v/v) and about 1% (v/v), between about 0.1% (v/v) and about 1% (v/v), between about 0.1% (v/v) and about 0.5% (v/v), between about 0.1% (v/v) and about 0.4% (v/v), and between about 0.2% (v/v) and about 0.5% (v/v). In some instances, the surfactant can be added at a concentration of about 0.4% (v/v). The ink may be printed on most planar surface, including paper, such as a commercial glossy paper. The ink is printed on the planar surface to form all three electrodes (e.g. reference, working and counter electrodes) in the set. All electrodes in the set may be formed of the same material or different materials. Typically, all the electrodes in the set are formed of the same conducting polymer. All electrodes can be printed in a single step.

To insulate/separate the sensing area from the contact areas, one, two, three, or more layers of dielectric ink may be printed on top of the electrodes. In some instances, the dielectric ink is printed over a surface of at least one of the electrodes in a set of electrodes. In some instance, the dielectric ink is printed over a surface of all three electrodes in a set of electrodes. In some instances, the dielectric ink is printed over the electrical interconnects of the working, reference, and counter electrodes. Typically, the dielectric ink is UV-curable.

For the biofunctionalization of the sensor, a biological ink containing a mediator (e.g. ferrocene), a polymer matrix, (e.g., chitosan, a polymer for forming a biocompatible matrix and entrapping the mediator in a polymeric biocompatible matrix) and a biofunctional molecule, i.e. a specific enzyme (e.g. glucose oxidase), or a mixture of enzymes, is printed on top of the working electrode to form a biofunctional coating. The biofunctional molecule may be immobilized on or in the polymer matrix via non-covalent or covalent bonding, such as via chemical conjugation, e.g., EDC-NHS coupling reaction where carboxyl groups of the enzyme may be conjugated to the amine groups of the polymeric matrix. In some instances, both the mediator and the biofunctional molecules are physically entrapped in the polymer matrix. In some instances, the biofunctional molecules are covalently immobilized on or in the polymer matrix and the mediator is physically entrapped in the polymer matrix. This typically forms the biofunctional coating of the working electrode.

A protective coating may be applied onto the electrodes, including onto the biofunctional coating, by printing a coating polymer on top of the electrodes. The protective coating may be printed on the entire surface of the electrodes, including on the biofunctional coating of the working electrode, or on a portion of the electrodes and on a portion of the biofunctional coating of the working electrode. In some instances, the protective coating is printed on the active areas of the working, reference, and counter electrode. In some instances, the protective coating is printed on the active area of the working electrode. In some instances, the protective coating is printed on the biofunctional coating of the working electrode.

For example, the coating polymer or a polymer mixture, such as a mixture containing Nafion® may be printed on top of the sensing area (comprising the active areas of the working, counter and reference electrodes) to block the interferences present in biologic milieu/media such as saliva or sweat.

An exemplary method for making and calibrating a sensor for detecting glucose is presented in Example 1.

An acquisition system, such as a potentiostat, is commercially available. It may be attached to the sensor by connecting each electrode to a lead in the acquisition system.

The acquisition system may then be connected to a display system, such as a device with a display screen. Exemplary display systems include smartphones, tablets, laptops, desktops, and smartwatches, are commercially available. The display systems typically include electronic conversion means, such as software, to convert the signals received from the acquisition system to a concentration value or a graph, which is then displayed on the screen. Such conversion means are known in the art.

### IV. Methods of Using the Sensors

The sensor system may be portable, wearable, or attachable to a subject. In some aspects, the sensor is small enough to be applied onto a medical device or onto a subject. The sensor's backing layer may be a planar surface, such as a paper, a tape, a bandage, a catheter, a lens, a patch, an implant, or a pad. The sensor, therefore, may be part of a catheter, a contact lens, a medical implant. The sensor may be worn by a subject as a patch or on a bandage, or may be provided in a kit, ready to be used as needed.

The sensor may be connected to an acquisition system, such as a potentiostat, and, optionally, to a display system. The display system may be a portable display system with a screen to display sensor reading. Portable display systems include smartphones, tablets, laptops, desktop, pagers, watches, and glasses.

An exemplary method of use includes applying a test sample onto the sensing area of the sensor, and obtaining a reading indicating that a metabolite is detected. Optionally, a polymeric well is used on top of the sensing area of the sensor to confine the test sample. Alternatively, if an acquisition system and/or a portable system is used, the method may include also obtaining a concentration of the metabolite of interest in the sample.

The information obtained from the sensors or sensor systems may be used to guide treatment of a disease or provide diagnosis of a disease.

A. Subjects

Generally, a subject is a mammal or bird providing a sample for measuring or detecting a metabolite within the sample. The subject may be in need of diagnosis of a disease, or in need of monitoring a treatment outcome for a disease.

A subject may be a control subject providing a control sample. The control subject may be a known or suspected case of a disease.

B. Test Samples

Typically, the sensor permits non-invasive testing of the presence, absence, or concentration of, a biological molecule in a test sample. The test sample can be a buffer solution, a biological sample, or a combination of both. Exemplary buffer solutions include phosphate buffer solution (PBS), salt water, MES buffer, Bis-Tris buffer, ADA, ACES, PIPES, MOPSO, Bis-Tris propane, BES, MOPS, TES, HEPES, DIPSO, MOBS, TAPSO, Trizma, HEPPSO, POPSO, TEA, EPPS, Tricine, Gly-gly, Bicine, HEPBS, TAPS, AMPD, TABS, AMPSO, CHES, CAPSO, AMP, CAPS, CABS, or a combination thereof. The buffer solution can have a pH between 3 and 8.5. In Typically, the buffer solution has a pH of 7.4. Exemplary biological samples include bodily fluids such as such as saliva, sputum, tear, sweat, urine, exudate, whole blood, serum, plasma, fecal sample, mucus or vaginal secretion. The biological molecules may be biomarkers, metabolites, or a combination thereof. Exemplary biological molecules that may be detected with the sensors include glucose, glucose-1, D-glucose, L-glucose, glucose-6-phosphate, ammonia, methanol, ethanol, propanol, isobutanol, butanol and isopropanol, allyl alcohols, aryl alcohols, glycerol, cholesterol, propanediol, mannitol, glucoronate, aldehyde, carbohydrates, lactate, lactate-6-phosphate, D-lactate, L-lactate, fructose, galactose-1, galactose, aldose, sorbose, mannose, glycerate, coenzyme A, acetyl Co-A, malate, isocitrate, formaldehyde, acetaldehyde, acetate, citrate, L-gluconate, beta-hydroxysteroid, alpha-hydroxysteroid, lactaldehyde, testosterone, gluconate, fatty acids, lipids, phosphoglycerate, retinal, estradiol, cyclopentanol, hexadecanol, long-chain alcohols, coniferyl-alcohol, cinnamyl-alcohol, formate, long-chain aldehydes, pyruvate, butanal, acryl-CoA, steroids, amino acids, favin, NADH, NADH₂, NADPH, NADPH₂, and hydrogen. In a preferred embodiment, the metabolite is glucose, glucose-1, D-glucose, L-glucose, glucose-6-phosphate, cholesterol, nicotine, carbon monoxide, and infectious agent metabolites. In some instances, the metabolite to be detected is glucose.

Typically, the volume of test sample for measurement can be between about 0.1 μL and about 1 mL. In some instances, the volume of test sample is between about 0.1 μL and about 100 μL, between about 0.1 μL and about 50 μL, between about 0.1 μL and about 30 μL, between about 1 μL and about 30 μL, between about 10 μL and about 30 μL. In some instances, the volume of test sample is about 30 μL.

The sensors may be used to detect metabolites that help with diagnosing a presence or absence of a disease, such as metabolic disease such as diabetes, a malignant disease, neurological disease, alcoholism, infection (viral, bacterial or fungal), immune response (allergy, asthma, immunosuppression), and cardiovascular disease. The sensors may be used to detect metabolites that help with prognosis of a disease or a disease course, such as such as metabolic disease, diabetes, malignant disease, neurological disease, alcoholism, viral infections, bacterial infections, and cardiovascular disease.

C. Methods of Diagnosis or Monitoring of Disease

Typically, a disease is diagnosed or monitored by using the sensors to detect a given metabolite or other compound or molecule known to be a biomarker for that disease. The methods of diagnosis may uses sensors alone, or may use sensors in combination with other diagnostic methods, including, but not limited to, cytology, histopathology, non-invasive imaging, and/or clinical assessment, to diagnose a subject with a disease.

The method of diagnosis includes measuring the level of a metabolite known to a biomarker for a disease in a biological sample. The biological sample is typically obtained from a subject in need of diagnosis (test sample). The method may further include comparing the value obtained for the metabolite in the test sample to a value for the same metabolite in a sample obtained from a control subject (control sample). The values for the metabolite in the test sample and control sample may then be compared to determine if the test sample includes a lower value of a given metabolite than that for the control sample.

Alternatively, the method of diagnosis may include comparing the normalized intensity of the biomarker in the test sample to a reference value. For example, the reference value for a given biomarker can be provided as a chart, and an increase in the normalized intensity for the given biomarker may indicate presence of a malignant proliferative disease, such as a malignant pleural effusion.

1. Metabolites as Biomarkers of Disease

The metabolites detected by the sensors may be biomarkers for a disease, or progression of a disease.

Exemplary metabolites that may be biomarkers of carbohydrate metabolism dysfunction, including diabetes, include carbohydrates glucose, pyruvate, oxaloacetate, fructose-6-phosphate, acetyl coenzyme A (acetyl-CoA), oxoglutarate, 2-oxoglutarate, pentose phosphate, glucose 6-phosphate, ribulose 5-phosphate, ribose 5-phosphate, phosphoribosyl pyrophosphate, glyceraldehyde-3-phosphate, gluconate, glycerate-3-phosphate, Glycerol-3-phosphate, gluconate, galactonate, glycerate, propanoyl coenzyme A (propanoyl-CoA), galactose, alpha-D-glucose-1-phosphate, D-galactonate, D-glucose 1-phosphate.

Exemplary metabolites that may be biomarkers of lung disease include glutamine, methionine, valine, hypoxanthine, inosine, isoleucine, sphingosine, palmitoylcarnitine, lysoPC(18:2), C8-ceramide, linoleamide, lysoPC(22:5), lysoPC(20:3), and palmitic amide.

Exemplary metabolites that may be biomarkers of neurodegenerative diseases, such as Alzheimer's disease (AD), Parkinson's disease (PD), and amyotrophic lateral sclerosis (ALS), include uric acid, choline, creatine, L-glutamine, alanine, creatinine, and N-acetyl-L-aspartate.

Exemplary metabolites that may be urinary biomarkers of a disease, such as infections, include tyrosinamide, biotin sulfone, hexanoic acid, 1-aminonaphthalene, 7-dehydrocholesterol, and azelaic acid.

Exemplary metabolites that may be biomarkers of a proliferative disease include acetone, 3-hydroxybutyrate, 1-methylhistamine, 1-methylnicotinamide, 2-methylglutarate, 2-oxoglutarate, 3-OH-3-methylglutarate, 3-methyladipate, 4-aminohippurate, acetone, adenine, alanine, creatine, dimethylamine, formate, fumarate, glucose, glycolate, imidazole, lactate, methylamine, O-acetylcarnitine, oxalacetate, phenylacetylglycine, phenylalanine, tryptophan, tyrosine, cis-aconitate, myo-inositol, trans-aconitate, leucine, valine, acetate, acetoacetate, creatinine, and trimethylalanine-N-oxide.

Exemplary metabolites that may be biomarkers of a cardiovascular disease include cholesterol, nicotine, carbon monoxide, nitrite, nitrate, alcohol gamma-aminobutyric acid (GABA), uric acid, citric acid, hypoxanthine, and inosine.

### V. Kits

Kits containing sensors for testing biological samples of a subject, and, optionally, one or more containers with buffers for preparing the samples for detection also provided herein. The kits can also include an instruction manual for sampling and detection of the one or more metabolites. Kits may also include instructions on instrument and/or software settings for calibrating and detecting the metabolite concentration.

The present invention will be further understood by reference to the following non-limiting examples.

### EXAMPLES

### Example 1. Printing the Glucose Sensor with Inkjet Printer

Materials and Methods for Examples 1-4

Inks formulation: To formulate the PEDOT:PSS ink, a solution including PEDOT:PSS dispersion (Heraeus, CLEVIOS™PJET700 N), 1 wt % glycidoxypropyltrimethoxysilane (GOPS, Sigma Aldrich), and 0.4% v/v of dodecyl benzene sulfonic acid (DBSA) was prepared, GOPS was added to prevent any delamination of the conducting pattern from the paper and DB SA to improve the wettability of the ink and film formation during printing. For the preparation of the biological ink containing the enzyme, 10 mg of glucose oxidase (from Aspergillus Niger>=100U mg⁻¹, Sigma Aldrich) was mixed in 0.1 M standard phosphate buffer solution (PBS, Sigma Aldrich). The enzyme solution was then mixed with EDC:NHS (1:1) 200 mM in 2-(N-morpholino)ethanesulfonic acid (MES) buffering agent in a 5:1:1 ratio. EDC:NHS solution was prepared by first addition of EDC and 30 min after this, including NHS in the reaction mixture. 28 mg Chitosan (from Shrimps, Sigma Aldrich) was dissolved in 0.2M acetic acid. 2.3 mg/mL of Ferrocene (Sigma Aldrich) solution was prepared in ethanol and left for 30 min in an ultrasonic bath. These two solutions were then mixed thoroughly for 30 min Once the Chitosan/Fc solution was ready, it was mixed with the enzyme activated solution and left overnight in the fridge at 4° C. EDC-NHS reaction conjugated the carboxyl groups of GOx to the amine groups of chitosan. For the preparation of the anti-interference layer, Nafion® (Nafion® 117 solution, Sigma Aldrich) was mixed with deionized water at a ratio 4:1 and its final concentration was 1 wt %. A commercial dielectric ink (EMD6200, SunChemical) was used for the fabrication of the insulation layer.

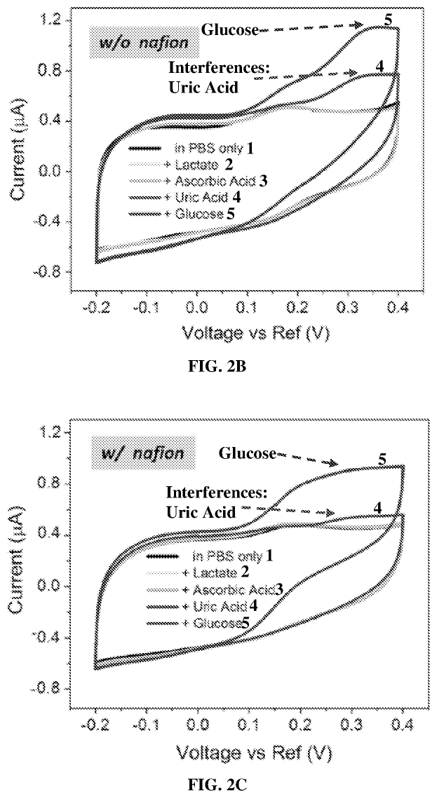

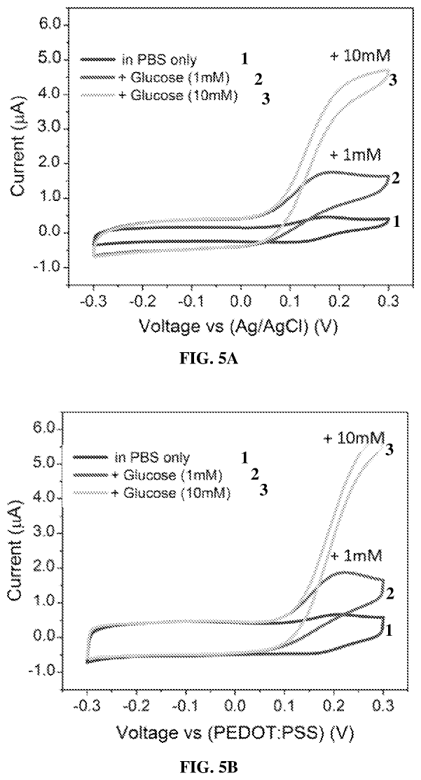

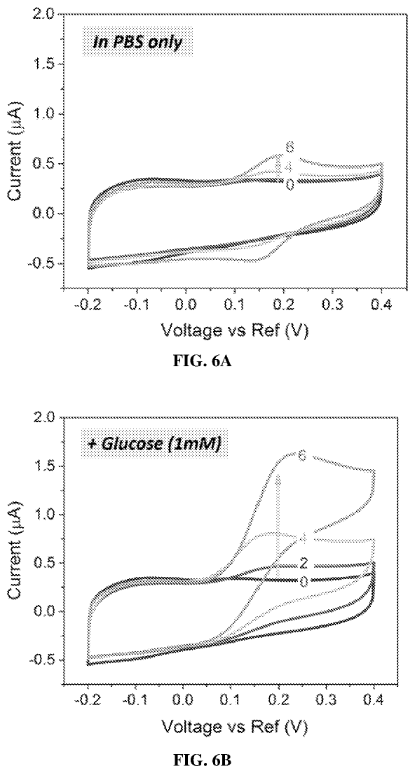

Ink jet printing: A Dimatix DMP-2800 inkjet printer was used to fabricate the device. 2 layers of PEDOT:PSS ink was printed on a commercial glossy paper (ArjoWiggins). The dimensions are shown in FIGS. 5A and 5B. The drop spacing was 20 μm. Following printing, the samples were cured for 30 min at 160° C. in a conventional oven. The electrical characterization of a 1 cm² printed PEDOT:PSS square was conducted using a four point probe system (Jandel). The second printing step was for casting of the dielectric layer to insulate the PEDOT:PSS areas outside of the sensing and the connection areas. Upon printing 1 layer of the dielectric ink, the pattern was cured for 5 min in a UV/Ozone chamber (Ossila, UV ozone cleaner). The biological ink containing enzymes (2, 4, 6, layers) were printed and let dry at room temperature for 24 h. Finally, NAFION® (sulfonated tetrafluoroethylene based fluoropolymer-copolymer) was printed on top of the sensing area (formed of the reference, working, and counter electrodes, see FIGS. 1A-1C and 2A-2C) to prevent the interferences coming from saliva during the detection of glucose. To verify the accuracy of the PEDOT:PSS electrodes for biosensing, 2 layers of silver were also printed as reference and counter electrodes (ink DGP HR, ANP.Co). The silver coatings were treated by a bleach solution (Clorox, 10%) drop casted on the patterns to form Ag/AgCl electrodes. The CVs are shown in FIGS. 6A and 6B.

Scanning electron microscopy (SEM): SEM images of printed sensor layers were acquired using FEI Nova nano microscope at accelerating voltages of 3 kV and working distances of 4.5 mm. The samples were coated with 3 nm thick iridium and mounted on aluminum stubs using aluminum tape for imaging. FIB-SEM for cross-section image was prepared on an FEI Helios NanoLab 400 S FIB/SEM dual-beam system equipped with a Ga+ ion source. C/Pt layers were deposited on the surface region of interest by Electron & Ion beam for sample protection.

X-ray photoelectron spectroscopy (XPS): XPS experiments were performed on a KRATOS Analytical AMICUS instrument equipped with an achromatic Al Ka X-ray source (1468.6 eV). Typically, the source was operated at voltage of 10 kV and current of 10 mA generating 100 Watts. The high-resolution spectra were acquired using a step of 0.1 eV. The pressure in the analysis chamber was in the range of ×10⁻⁷ Pa during the whole measurement time.

Device characterization: Cyclic voltammetry (CV) and chronoamperometry measurements were performed using a potentiostat-galvanostat (Metrohm Autolab B.V.) and the data were collected with NOVA software. CV scans were recorded in PBS solution from −0.2 to 0.4 V vs. PEDOT:PSS reference electrode, unless otherwise stated. For chronoamperometry measurements, the voltage was set at 0.25V vs. reference electrode. All the data shown in this work were collected from devices that were used 24 h after being printed unless stated otherwise.

Chronoamperometric measurements for the calibration of the sensor: To acquire the calibration curves, the applied potential was set to 0.25 V vs. reference electrode and interval time for data collection was 0.1 s. A PDMS well was placed on top of the active area (4×4 mm2) to confine the electrolyte. The total volume of the PBS solution in this well was 30 μL. Different concentrations of glucose (Sigma Aldrich) in PBS were added to this solution at a 1:10 ratio of the total volume. The glucose concentration varied between 25 μM and 2.6 mM. The response of three different devices (current-time curves) was measured to each added concentration of glucose. During the calibration measurements, it was ensured that the baseline current (in PBS, no glucose) was stabilized before the addition of glucose.

For the experiments using saliva, the saliva of a healthy volunteer was collected after fasting (12 h) and filtered the solution with a filter of 1 μm pore size. The glucose concentration in this sample was measured using a commercial Glucose Assay Kit (GAGO-20, Sigma Aldrich) using a spectrophotometer (Promega). To mimic variations of glucose in physiological saliva, different concentrations of glucose were added to this sample and measured by the printed sensor. All protocols and procedures involving human saliva were approved by the KAUST Institutional Biosafety and Bioethics Committee (IBEC). The volunteers provided signed consent to participate in the study.

Results

For the fabrication of the device, a commercially available PEDOT:PSS ink dedicated for inkjet was selected and the ink formulation was further optimized to meet the substrate requirements for the formation of a uniform and conducting layer on paper. The ink was printed on a commercial glossy paper (ArjoWiggins) (used as backing 12) as shown in FIGS. 1A and 1C along with the printed PEDOT:PSS features. A three electrode, i.e. reference, working, and counter, cell configuration was used to measure the concentration of glucose present in the biological media. Current electrochemical sensors for metabolite typically use Ag/AgCl and platinum electrodes as reference and counter electrodes, respectively. Here, all the electronic components including the contact pads (70) of the electrochemical system (e.g., reference (10), working (20), and counter (30) electrodes) were composed of the same material, conducting polymer PEDOT:PSS (FIGS. 1B, 1C, and 1E). The electrical conductivity of the conducting polymer ink was found to be 250 S/cm.

To insulate/separate the sensing area 40 (containing reference (10), working (20), and counter (30) electrodes; the working electrode (20) containing an enzyme 54 and a mediator 56 covered with Nafion® 58) from the contact pads area (70), one layer of UV-curable dielectric ink (52) was printed on top of the electrode interconnects as described in FIGS. 1C and 1E.

For the incorporation of the biorecognition element, a biological solution containing both the mediator 56 (i.e. ferrocene (Fc) complex) and a specific enzyme Glucose Oxidase (GOx) (54) known for its utilization/use in the determination of glucose in body fluids, was printed on top of the working electrode 20 (see experimental section for details). Fc is an electron mediator commonly used in enzymatic sensors as a co-substrate to replace oxygen. It molecularly wires the enzyme to the sensing electrode, therefore improves the selectivity as well as the operation window of the sensor. However, as Fc adsorbs weakly onto surfaces by itself, its leakage can raise toxicity concerns. Fc in a solution was mixed with the polysaccharide, chitosan. While entrapping Fc within its biocompatible matrix, chitosan improves the processability of the enzyme. The resulting ink was printed on top of the working electrode (20). The biological ink was immobilized on top of the conducting polymer via EDC-NHS coupling reaction where carboxyl groups of GOx were conjugated to the amine groups of chitosan.

Ultimately, a thin layer of Nafion® (58) was printed over the sensing area (40) containing the working (20), counter (30), and reference (10) PEDOT:PSS electrodes. As a polyanion, Nafion® acts as a barrier for the interfering species present in complex biological milieu or formed as a result of unspecific redox reactions during electrode operation (Yuan, et al., Electroanalysis, 17:2239-2245 (2005)).

The cross sectional SEM image of a typical working electrode shows incorporation of all the vertical layers of the sensor where PEDOT:PSS, biological coating and Nafion® layer have a thickness of 160 nm, 655 nm, and 190 nm, respectively. As the working electrode is built as a layer-by-layer assembly, the morphology and chemical composition of each layer was examined. While the surface of PEDOT:PSS film on paper is relatively featureless, upon the addition of the biological ink and thereafter of Nafion®, the surface microstructure undergoes large changes. High resolution X-ray Photoelectron Spectroscopy (XPS) C 1s spectra show characteristics peaks representative of each layer: for instance, C—O for PEDOT:PSS, C═O for the biological ink (chitosan) and C—F for Nafion®. The peaks located in N is region only appear after the biological layer is printed. S 2p spectrum undergoes changes upon Nafion® addition due to the SO₃ bonds, confirming the layer-by-layer integration of each component on top of PEDOT:PSS.

### Example 2. Testing the Operability of the Printed Sensor

Materials and Methods

The materials and methods used for testing are presenting in Example 1.

Results

The mechanism of glucose detection based on the enzyme/mediator complex involves a cycle of electrochemical reactions at the surface of the working electrode (20), as depicted FIG. 1D. Upon reacting with glucose, GOx gets reduced. The reduced enzyme cycles back via the ferrocene/ferricenium (Fc/Fc⁺) ion couple which mediates electron transfer from the active sites of GOx to the underlying PEDOT:PSS electrode. This reaction causes a change in the current flowing between the working (20) and counter (30) electrodes, proportional to the concentration of glucose, which are detected by the acquisition system. To transfer the data to a portable system, such as a smartphone or a tablet, the biosensor is connected to a miniaturized portable acquisition system.

A different number of layers, i.e., 2, 4 and 6 layers, of the biological ink containing enzyme/chitosan/ferrocene were printed, and the CVs recorded for each device were compared. As shown FIG. 6A, the anodic and cathodic peaks were clearly distinguishable in the CVs demonstrating the presence and the entrapment of ferrocene in the chitosan matrix, resulting in an electrochemical reaction between the conducting polymer and the mediator. A direct correlation between the number of printed layers with the amplitude of Fc peaks was observed. In response to the addition of glucose in the media, the anodic peak increased significantly accordingly with the number of printed layers (FIG. 6B). To characterize the device, cyclic voltammetry in the potential range from −0.2V to 0.4V was performed. The scan rate was 20 mV/s and it was chosen to print 6 successive layers of the biological ink for the rest of the work to test the sensitivity of the devices.

FIG. 2A shows the CV response of the sensor before and after its modification with the Fc/GOx film, as well as in the presence of different concentrations of glucose (respectively 1 mM and 10 mM) in Phosphate Buffer Saline (PBS), a standard buffered solution commonly used in biological research. The electrolyte, phosphate buffered saline solution (PBS, pH 7.4) is placed on top of the active area of the sensor. The well-defined and symmetric peaks at ca. 0.2 V and ca. 0.15 V (anodic and cathodic, respectively) of the biofunctionalized PEDOT:PSS are characteristic of Fc (FIG. 2A). Upon addition of glucose into the PBS solution, we observe a drastic increase in the anodic current, evidencing effective immobilization of GOx, its reaction with glucose and communication with Fc and PEDOT:PSS (FIGS. 2A and 6B). The current increase further with more glucose present in the solution. This change in the current, was in accordance with the assumptions of Yun et al., Anal. Sci. Int. J. Jpn. Soc. Anal. Chem. 27:375 (2011). However, Yun et al. only printed a working electrode and some biological components employed in the electrochemical reaction. External Pt and Ag/AgCl electrodes were used in Yun's system as counter and reference electrodes, respectively. In this work, all the electrodes necessary to operate the sensor are made of PEDOT:PSS printed at a single step, as well as other components printed in an automated fashion.

To evaluate the performance of PEDOT:PSS as a reference electrode, we measured the open circuit potential of a PEDOT:PSS film vs. printed PEDOT:PSS reference electrode in PBS and saliva as a function of time. The potential of the electrode quickly stabilized in both media and remained constant over the course of the measurement. To verify the accuracy of the all-polymer system, another configuration was tested that includes counter and reference electrodes printed using a commercially available silver ink and post treated with a bleaching solution for chlorination to create Ag/AgCl electrodes. Then, the same experiments (CVs) were performed on both configurations (Ag/AgCl as counter and reference electrodes (FIG. 5A) and PEDOT:PSS only for the second configuration (FIG. 5B). Almost identical responses in the CVs were observed when glucose was added in PBS showing the accuracy of the system composed only of PEDOT:PSS electrodes.

In more complex biological fluids such as saliva, many molecules are present and are known to interact with the conducting polymer due to their electroactive nature. These interferents are the main obstacles in electrochemical detection of glucose as they lead to inaccurate read-outs. For example, uric acid and ascorbic acid have their oxidation potentials within the operation potential of our sensor (V=0.35 V) and can thus be oxidized by PEDOT:PSS. In order to overcome the interference, cation exchange membranes containing materials like chitosan and Nafion® are typically coated on top of sensing electrodes as they prevent negatively charged species from reaching the electrode surface (Jia et al., Anal. Chem. 85:6553-6560 (2013); Lee et al., Sci. Adv. 3:e1601314 (2017); and Sempionatto et al., Lab. Chip 17:1834-1842 (2017)). Without such an encapsulation layer, the device is sensitive to the most common interfering compounds, i.e., lactate, ascorbic acid and uric acid, all introduced to the measurement solution in the concentration range relevant to their physiological levels in saliva, that is 2 mM, 0.01 mM, and 0.15 mM, respectively (Pappa, et al., Adv. Healthc. Mater., 5:2295-2302 (2016); Makila, et al., Arch. Oral. Biol., 14:1285-1292 (1969); and Inoue, et al., J. Chromatogr. B. Anal. Technol. Biomed. Life Sci., 785:57-63 (2003)).

As shown in FIG. 2B, among the three molecules introduced in the PBS, the sensor was most sensitive to uric acid. In the presence of uric acid, the CV exhibited an increase in the anodic peak in the same potential range as for glucose detection, while the response to lactate or ascorbic acid is rather negligible. Without a barrier layer, this electrochemical reaction would result in a false detection of glucose by the printed sensor once tested in a complex media such as saliva. The presence of a 190 nm thick (2 printed layers) Nafion® membrane on top of the biological coating eliminates the diffusion of uric acid and reduces the current response by 84% (FIG. 2C).

### Example 3. Testing the Sensitivity and Reusability of the Printed Sensor

Materials and Methods

The materials and methods used for testing are presenting in Example 1.

Results

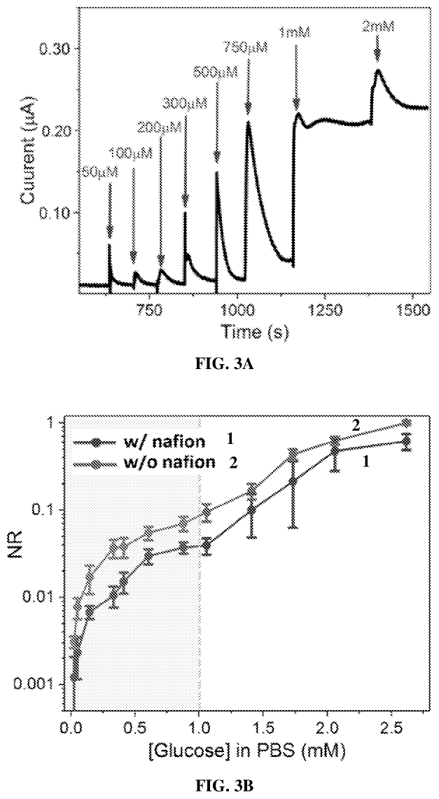

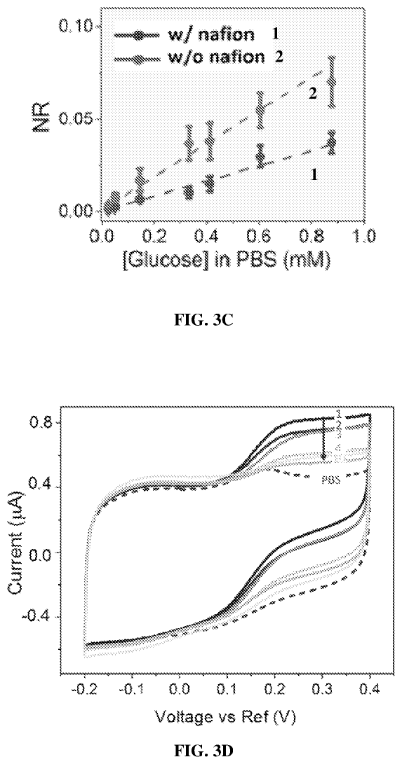

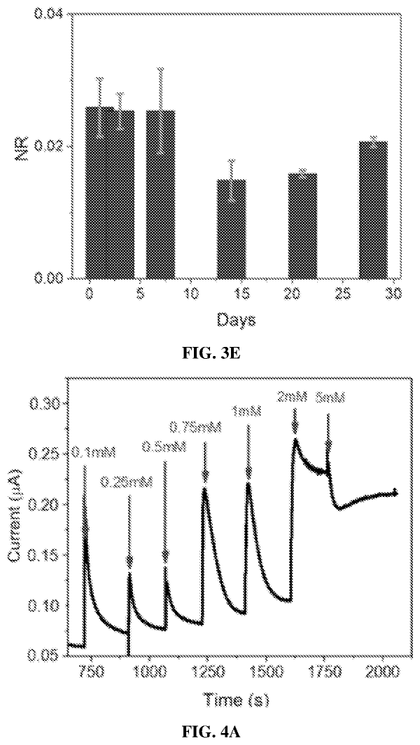

In order to evaluate the sensor performance, the real-time changes in the current of the sensor upon additions of cumulative concentrations of glucose from 50 μM to 2 mM into the measurement solution at a potential of 0.25 V vs. the PEDOT:PSS reference electrode were recorded (FIG. 3A). The measurements were performed in presence and absence of the Nafion® membrane and repeated the experiments to verify the accuracy of the sensor. As shown in FIG. 3A, the current increased with the additions of glucose corresponding to the productions of electrons generated by the electrochemical reactions.

To account for batch-to-batch variations in the current output of the sensors, the current response to glucose was normalized with the read-out signal at zero glucose concentration and the maximum possible output of the device. The normalized response (NR) of the sensor is calculated from:

$$NR = \frac{( {I}_{d} - {I}_{0} )}{( {I}_{m} - {I}_{0} )} \qquad Equation ( 1 )$$

where I₀ is the baseline current (i.e., the current measured after stabilization of the sensor without glucose), Iₘ is the maximum possible current that the readout can reach (i.e., saturation), and I_{d} is the current measured at a given glucose concentration. Note that I_{d} reaches as stable value ca. 60 s after the addition of glucose, which gave the extraction of a calibration curve.

As depicted in FIGS. 3B and 3C, the NR of the sensor varied as a function of glucose concentration. For concentrations between 25 μM and 0.9 mM, the current increased linearly and the sensor reached a plateau after the introduction of 2.5 mM of glucose. The presence of the Nafion® membrane somehow hindered the interactions between glucose in PBS and the biological coating, resulting in a reduced NR but overall exhibited a similar saturation regime with a linear response to concentrations lower than 0.9 mM (FIGS. 3B and 3C). FIG. 3C shows the normalized response linearly with the variation of the concentration of glucose.

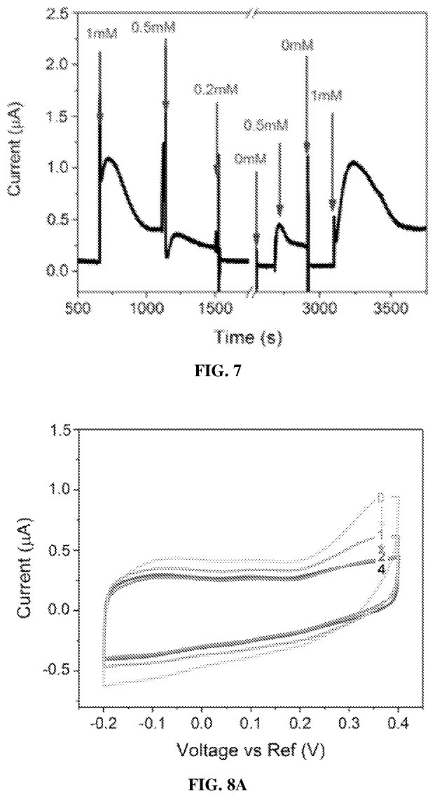

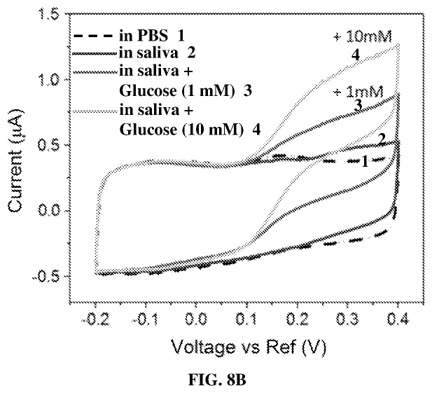

Increasing the thickness of the encapsulation layer impedes the diffusion of molecules into the PEDOT:PSS layer underneath, while a difference between 2 or 4 layers of Nafion® was not observed (FIG. 8A). Although the efficacy of Nafion® in eliminating interference comes at the expense of sensitivity and speed, both the steady-state and the time resolved characteristics of the sensors are within sufficient boundaries. The linear range of this sensor corresponds to the physiological levels of glucose in saliva, typically ranging between 20 μM and 1 mM (FIG. 3C).

To test the reusability of these sensors, CVs were recorded in the presence of glucose including several washing steps with PBS. For these measurements, one cycle includes exposing the sensor to PBS, addition of glucose (1 mM) and then replacing the glucose solution with fresh PBS. Until fourth cycle, a stable and accurate response to glucose (with 6% error) was obtained, while upon the fourth cycle, the amplitude of the oxidation peak is reduced by 45% (FIG. 3D). Between the fourth and tenth cycles, small changes in the peak amplitude were observed.

To evaluate the shelf life of the device over time, the performance of devices which have been stored for 24 h up to 1 month after their fabrication was tested. Once printed, these devices were stored in sealed vacuum bags at room temperature. The response of the sensors, i.e. peak current measured at 0.25 V, to 1 mM glucose was recorded (FIG. 3E). A slight reduction of the performance occurs 14 days after the device is fabricated. After one month, 80±3% of the NR recorded at the first day is maintained. Note that when the sensors were kept in the fridge at 4° C., no significant enhancement of shelf life was attained.

### Example 4. Measuring Glucose Levels in the Saliva of Healthy Subjects

Materials and Methods

The materials and methods are presenting in Example 1.

Results

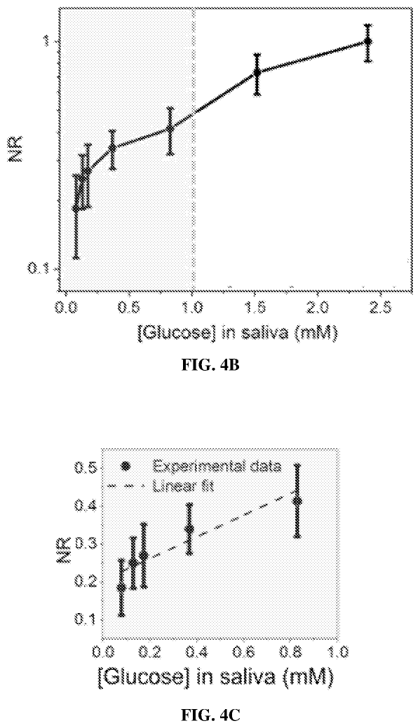

The printed sensor was tested with bodily fluid using saliva as the media. To that end, a sample of the saliva of a healthy non diabetic volunteer was collected, who was asked to fast 12 h before obtaining the oral fluid. The glucose in this sample was found at a concentration of 28 μM using a commercial Glucose (GO) assay kit (Sigma Aldrich). The CV curve of the sensor differs when measured in saliva compared to PBS due to the presence of glucose and other interferents (FIG. 8B). As the concentration of glucose in this biological sample was low, it was decided to use this sample as a buffer solution for the calibration of the sensor in saliva. The saliva was enriched and added glucose to mimic the glucose concentration range typical for diabetic patients (Abikshyeet, et al., Diabetes Metab. Syndr. Obes., 5:149-154 (2009)). The chronoamperometric signals of the device were recorded in response to cumulative additions of glucose, as depicted FIG. 4A. The device has a linear response to glucose within the range relevant to the glucose concentrations of non-diabetic and diabetic saliva (from 28 μM to 0.85 mM) (FIGS. 4B and 4C) (Abikshyeet, et al., Diabetes Metab. Syndr. Obes., 5:149-154 (2009); Kumar, et al., Contemp. Clin. Dent., 5:312 (2014); Gupta, et al., J. Oral Maxillofac. Pathol., 21:334-339 (2017); and Naing, et al., J. Diabetes Metab. Disord., 16:2251-6581 (2017)). Diabetic patients are advised to keep their blood glucose levels close to the target range below 7 mM (fasting) (Wustoni, et al., Adv. Mater. Interfaces, (2018); The Global Diabetes Community, http://www.diabetes.co.uk/diabetes_care/blood-sugar-level-ranges.html.). Studies have demonstrated a significant positive correlation between the concentration of glucose in saliva and blood for healthy and diabetic patients and substantiated the role of saliva as a noninvasive diagnostic tool (Abikshyeet, et al., Diabetes Metab. Syndr. Obes., 5:149-154 (2009); Kumar, et al., Contemp. Clin. Dent., 5:312 (2014); Gupta, et al., J. Oral Maxillofac. Pathol., 21:334-339 (2017); and Naing, et al., J. Diabetes Metab. Disord., 16:2251-6581 (2017)). In saliva, the equivalent of this concentration is ca. 0.13 mM (Abikshyeet, et al., Diabetes Metab. Syndr. Obes., 5:149-154 (2009)). As such, the sensor is relevant for diabetes treatment and reducing the risk of a coma. The sensor response is modulated only by the dose, it is reversible and independent of how glucose was introduced into the solution: the device showed the same read-out to a particular glucose concentration regardless of whether it is exposed to first low or high concentrations of glucose (FIG. 7). For daily use, the paper-based electronics can be easily integrated with a portable miniaturized measurement system wherein the sensor is placed and electrically contacted without any wires. The system then transfers the read-outs wirelessly to a smartphone or a tablet which correlates the current value to glucose concentration.

The Examples show that a process such as inkjet printing is compatible and can be used with inexpensive, eco-friendly, recyclable, and flexible substrates (such as paper) to form non-invasive, pain-free, accurate, needle-free sensors for daily monitoring of a metabolite, such as glucose, from biological media such as saliva. This is achieved by fully printing the electrodes using the same material, a biocompatible conducting polymer PEDOT:PSS, and simply functionalizing the working electrodes. All the components of this sensor were printed as a layer-by-layer assembly, including the conducting polymer as the electronic component, a biological film containing the enzyme/mediator as well as a dielectric and encapsulation layer. This sensor shows long term stability as it was successfully testes over a period of 1 month. The sensor has high sensitivity in the relevant range of glucose in saliva. Forming such versatile and multifunctional biomedical platform provides a step forward for wearable biomedical devices that are user-friendly, automated and financially affordable.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A sensor for detecting a biological molecule, the sensor comprising
(i) a backing layer with a first surface; and
(ii) a set of electrodes printed on the first surface of the backing layer; and optionally
(iii) a data acquisition system,
wherein the set of electrodes comprises a reference electrode, a working electrode, and a counter electrode, and
wherein the electrode comprise a conducting material.

2. The sensor of claim 1, wherein: (a) the sensor comprises more than one set of electrodes, and wherein each electrode of the set of electrodes comprises an active area, an electrical interconnect, and a contact area; (b) the conducting material is a conducting polymer; (c) the working electrode comprises a mediator and a biofunctional molecule; and (d) wherein the backing layer is any layer with a planar surface selected from the group consisting of a paper, a tape, a tattoo, a bandage, a catheter, a lens, a patch, an implant, and a pad.

3. (canceled)

4. (canceled)

5. (canceled)

6. The sensor of claim 2, wherein the mediator and the biofunctional molecule are entrapped in a polymer matrix and optionally, wherein the polymer matrix comprises a positively charged polymer.

7. The sensor of claim 6, wherein the polymer matrix is positioned over the active area of the working electrode.

8. The sensor of claim 1 comprising: (a) a dielectric coating wherein the dielectric coating is positioned on the electrical interconnects of the set of electrodes; (b) a sensing area comprising the active areas of the reference electrode, the working electrode, and the counter electrode, wherein the sensing area optionally comprises a protective coating comprising a synthetic ionic polymer selected from the group consisting of polystyrene sulfonate, and perfluorinated sulfonated ionomers.

9. (canceled)

10. (canceled)

11. (canceled)

12. The sensor of claim 2, wherein: (a) the conducting polymer is a polymer selected from the group consisting of poly(4,4-dioctylcyclopentadithiophene), poly(isothianapthene), poly(3,4-ethylenedioxythiophene), polyacetylene (PAC), polyaniline (PANI), polypyrrole (PPY) or polythiophenes (PT), poly(p-phenylene sulfide) (PPS), and poly(3,4 ethylenedioxythiophene) polystyrene sulfonate (PEDOT:PSS); and/or the mediator is selected from the group consisting of multivalent metal ions, organometallic compounds, phenazine methosulfate, dichlorophenol indophenol, short chain ubiquinones, ferrocene complex, co-factors, or a combination thereof; and/or wherein the biofunctional molecule is selected from the group consisting of carbohydrates, peptides, proteins, and nucleic acids, and optionally, is an enzyme.

13. (canceled)

14. The method of claim 12, wherein the mediator is a ferrocene complex

15. (canceled)

16. (canceled)

17. (canceled)

18. The sensor of claim 6, wherein the positively charged polymer is selected from the group consisting of alginate amine, chitosan, dextran amine, heparin amine, and a combination thereof.

19. (canceled)

20. (canceled)

21. The sensor of claim 1, further comprising an acquisition system and a display system.

22. The sensor of claim 21, wherein: (a) the acquisition system is a potentiostat: (b) the display system is a portable display system comprising a screen to display sensor reading, selected from the group consisting of smartphones, tablets, laptops, desktop, pagers, watches, and glasses.

23. (canceled)

24. (canceled)

25. A method of making a sensor of claim 1, the method comprising inkjet-printing a conducting polymer onto a backing layer and forming a set of electrodes; and optionally, wherein the electrodes are printed in one step.

26. The method of claim 25, wherein the set of electrodes comprises three electrodes with a shape having a length between about 2 mm and about 20 mm, a width between about 0.1 mm and about 2 mm, and a height between about 0.1 mm and about 2 mm.

27. (canceled)

28. The method of claim 25 further comprising: (a) inkjet-printing a dielectric coating over a surface of at least one of the electrodes in the set of electrodes; (b) inkjet-printing a biofunctional coating over the surface of working electrode; and/or (c) inkjet-printing a protective coating over a surface of at least one of the electrodes, or over the biofunctional coating.

29. (canceled)

30. (canceled)

31. A method of using the sensor of claim 1, the method comprising applying a test sample to the set of the electrodes of the sensor.

32. The method of claim 31, wherein the test sample is a bodily fluid or mucus and is selected from the group consisting of saliva, sputum, tear, sweat, urine, exudate, blood, plasma, and vaginal discharge.

33. (canceled)

34. (canceled)

35. The method of claim 33, wherein the biological molecule is selected from the group consisting of a biomarker or a metabolite.

36. The method of claim 35, wherein the biological molecule is a metabolite of an anabolic or catabolic pathway selected from the group consisting of carbohydrate and lipid metabolism, nucleotide and amino acid metabolism, and secondary metabolism.

37. The method of claim 33, wherein the biological molecule is a metabolite of an anabolic or catabolic pathway selected from the group consisting of carbohydrate and lipid metabolism, including central carbohydrate metabolism, fatty acid metabolism, lipid metabolism, lipopolysaccharide metabolism, glycan metabolism, glycosaminoglycan metabolism, sterol biosynthesis; nucleotide and amino acid metabolism, including purine metabolism, pyrimidine metabolism, serine and threonine metabolism, cysteine and methionine metabolism, branched-chain amino acid metabolism, branched-chain amino acid metabolism, lysine metabolism, histidine metabolism, aromatic amino acid metabolism, other amino acid metabolism, cofactor and vitamin biosynthesis, polyamine biosynthesis; and secondary metabolism, including aromatics degradation, and biosynthesis of secondary metabolites.

38. The method of claim 33, wherein the biological molecule is a metabolite selected from the group consisting of glucose, pyruvate, oxaloacetate, fructose-6-phosphate, acetyl coenzyme A (acetyl-CoA), oxoglutarate, 2-oxoglutarate, pentose phosphate, glucose 6-phosphate, ribulose 5-phosphate, ribose 5-phosphate, phosphoribosyl pyrophosphate, glyceraldehyde-3-phosphate, glycerol 3-phosphate, gluconate, glycerate-3-phosphate, gluconate, galactonate, glycerate, propanoyl coenzyme A (propanoyl-CoA), galactose, alpha-D-glucose-1-phosphate, D-galactonate, D-glucose 1-phosphate, cholesterol, nicotine, carbon monoxide, nitrite, nitrate, alcohol, and bacterial metabolite.
